(19) United States
(12) Patent Application Publication
(10) Pub. No.: US 2012/0203258 A9
(43) Pub. Date: Aug. 9, 2012

(54) BLOOD LANCET DEVICE INCLUDING ADJUSTMENT OF THE PRICKING DEPTH

(75) Inventors: Andreas Knie, Regensburg (DE); Marion Butz, Regensburg (DE)

(73) Assignee: GERRESHEIMER REGENSBURG GMBH, Regensburg (DE)

(21) Appl. No.: 13/262,510

(22) PCT Filed: Mar. 17, 2010
(86) PCT No.: PCT/EP2010/053426

§ 371 Date: Sep. 30, 2011

(30) Foreign Application Priority Data
Apr. 3, 2009 (DE) 10 2009 016 126.0
Jun. 18, 2009 (DE) 10 2009 025 443.9

(51) Int. Cl. A61B 17/32

(52) U.S. Cl. 606/182

(57) ABSTRACT

The invention relates to a blood lancet apparatus (101, 201, 301, 401, 501) with a base member (103, 203, 303, 403, 503) for the arrangement of apparatus components. These comprise a lancet-receiving device for holding at least one lancet element, which is surrounded at least locally by the base member (103, 203, 303, 403, 503), and a cap (102, 202, 302, 402, 502, 602, 702, 802, 902, 1102, 1202, 1302, 1402, 1502, 1602) coupled to the base member (103, 203, 303, 403, 503) for the arrangement of a setting wheel (104, 204, 304, 404, 504, 604, 704, 804, 1004) on a portion (626, 726, 826) of the cap (102, 202, 302, 402, 502, 602, 702, 802, 902, 1102, 1202, 1302, 1402, 1502, 1602) which is cylindrical at least in part, in order to set a plurality of exit lengths (L1, L2, L3) of the lancet element out of the blood lancet apparatus (101, 201, 301, 401, 501). In addition, the setting wheel (104, 204, 304, 404, 504, 604, 704, 804, 1004) is produced with an internally threaded portion (624, 724, 824, 1524) and the cylindrical portion (626, 726, 826) has an externally threaded portion (628, 728, 828, 1328, 1528, 1628), wherein the threaded portions (624, 724, 824, 1524, 628, 728, 828, 1328, 1528, 1628) are connected to one another functionally in such a way that the setting wheel (104, 204, 304, 404, 504, 604, 704, 804, 1004) is rotatable and adjustable in the longitudinal direction (L) of the base member (103, 203, 303, 403, 503). According to the invention the setting wheel (104, 204, 304, 404, 504, 604, 704, 804, 1004) is surrounded by arm-like segments (318a, 418a, 518a, 318b, 418b, 518b) extending in the longitudinal direction (L), in such a way that an actuation of the setting wheel (104, 204, 304, 404, 504, 604, 704, 804, 1004) required for setting the exit length is capable of being carried out only in the segment-free peripheral portions.

The invention relates to a blood lancet apparatus with setting of the pricking depth according to the preamble of claim 1. In the case of blood lancet apparatus or pricking aids it is known that in order to set the pricking depth adjustable stops are used in the front region of the blood lancet apparatus, against which a lancet holder which embraces the lancet strikes in a preferably spring-loaded pricking procedure in which the lancet is moved out of a casing of the pricking aid, it being possible for this stop to be altered so that the lancet moves out of the casing of the pricking aid to different degrees—depending upon the setting of the stop. The structures of pricking aids designed in this way with an integrated stop capable of being altered are frequently complex, however, and they require the stop or the stop element respectively to be fixed in a desired position to be altered.

In accordance with the publication EP 1810615 A1a blood lancet apparatus is likewise known, in which a cap element is capable of being screwed onto the front side of a casing designed in the manner of a pin, it being possible for this cap element to be turned by means of a thread in order to arrange an outlet opening—situated on the front side of the cap element—at a greater or lesser distance from the rest of the casing.

A cap element of this type, which is arranged in an integral manner on the casing and which completely embraces both the outlet opening and the lancet holder arranged in it in part, can be set only with difficulty by means of only one hand in a position with respect to the remainder of the casing, in particular when operating the blood lancet apparatus. As a result of changing the distance of the outlet opening with respect to the remainder of the casing, an exit of the lancet tip to a differing degree from the cap element—which is completely closed with the exception of the outlet opening—is made possible.

Accordingly, a device for setting the pricking depth of a blood lancet apparatus is to be made available, which permits a simple manipulation in the adjustability thereof even by means of only one hand and prevents unintentional adjustment of the pricking depth selected.

The object is attained according to the invention by a blood lancet apparatus in accordance with the subject of claim 1. A blood lancet apparatus according to the invention has a base member for the arrangement of apparatus components. In addition, the blood lancet apparatus comprises a lancet-receiving device for holding at least one lancet element, which is surrounded at least locally by the base member, i.e. for example the lancet element can be situated completely inside the base member or can project in part out of the base member or the base member has openings through which the lancet element emerges.

Furthermore, a cap coupled to the base member is provided for the arrangement of a setting wheel on a portion of the cap which is cylindrical at least in part, in order to set a plurality of exit lengths of the lancet element out of the blood lancet apparatus.

In this case a cap can be regarded as being an extension of the base member and it is joined to the latter in an integral manner or in a non-releasable or releasable manner respectively, for example by being placed on the base member.

The setting wheel has an internally threaded portion and the cylindrical portion is provided with an externally threaded portion, the threaded portions being connected to each other functionally in such a way that the setting wheel is rotatable and is adjustable in the longitudinal direction of the base member, i.e. the rotational movement of the setting wheel is coupled by means of the threaded portions to an displacement of the setting wheel in the longitudinal direction in such a way that a longitudinal displacement of the setting wheel is capable of being carried out at the same time by a rotation of the setting wheel.

According to the invention the setting wheel is surrounded by arm-like segments extending in the longitudinal direction, in such a way that an actuation of the setting wheel required for setting the exit length is capable of being carried out only in the segment-free peripheral portions. This is advantageous since a stop by which the movement of the fingers or thumb is restricted is provided by the segments, as a result of which a controlled and ergonomically advantageous setting of the exit length is carried out.

In a further preferred embodiment of the present invention the cap and/or the base member has or have a cross-section which in a first cross-sectional direction of the width-wise direction is greater than the diameter of the setting wheel and in a second direction of the depth direction is smaller than the cross-section in the first direction. This is advantageous since, as a result, the base member and/or the cap has or have a shape on the basis of which the blood lancet apparatus rests in the hand in a defined manner or is received in the hand in an intuitive manner by the patient and operating steps capable of being misunderstood are therefore eliminated.

In a further preferred embodiment of the present invention the cylindrical portion of the cap is situated between the two arm-like segments of the cap enclosing the portion in part. This embodiment is advantageous, since one slot, which is preferably designed in such a way that penetration by a finger or the thumb respectively into the slot is eliminated, is formed in each case by a segment and the setting wheel.

The degree of the enclosing of the setting wheel by the segments additionally allows a conclusion to be drawn on the maximum possible rotation of the setting wheel in one actuating step.

In a further preferred embodiment of the present invention the segments are made uniform. This is advantageous since the design and production of the cap as well as further developments are substantially simplified, since a larger number of similar parts or similar areas or portions respectively are present.

In a further preferred embodiment of the present invention the segments are arranged in an opposed manner with respect to the cylindrical portion. This is advantageous since the apparatus is designed in a relatively symmetrical manner or in a symmetrical manner, as a result of which the mounting of the cap can be carried out in different orientations and it is accelerated and simplified as a result.

In a further preferred embodiment of the present invention the setting wheel is longer than the segments or projects at least in part in the longitudinal direction with respect to the segments. This is advantageous since this ensures that the surface of the setting wheel, preferably the flat surface of the setting wheel which terminates the setting wheel towards the outside and which has the exit opening for the lancet element, can be arranged in a desired manner at a handling point or on a body respectively.

In a further preferred embodiment of the present invention a first positioning device and a further positioning device extend at least in part in the longitudinal direction, in which case one of the two positioning devices is provided on the cap and the other positioning device is provided on the setting wheel.

The first positioning device comprises at least one pin and the further positioning device is made corrugated, in particular serrated/tooth-like. The at least one pin is capable of being deflected, i.e. flexible, at least for a time. The bending of the pin is carried out in such a way that a first face of the pin is moved relative to a second face of the pin. This embodiment is advantageous since as a result of these arrangements it is possible in a simple and inexpensive manner for a ratchet mechanism to be provided which will allow a step-wise setting of the exit width of the lancet element from the blood lancet apparatus.

In a further preferred embodiment of the present invention the first positioning device surrounds at least in part the cylindrical portion or is formed on it respectively and it forms an external set of teeth of which the teeth have tooth flanks, of which the majority is formed substantially equal. In addition, at least one pin is formed on the setting wheel. This has the advantage that the set of teeth and the pin co-operate in a protected manner in a region bounded by the setting wheel.

In a further preferred embodiment of the present invention the first positioning device is surrounded at least in part by the setting wheel. The setting wheel has at least in part an internal set of teeth and the teeth have tooth-flanks, of which the majority is formed substantially equal and which are inclined with respect to the radial direction extending from a centre axis of the cylindrical portion. In addition, a pin is framed on the—at least in part—cylindrical portion, and in a particularly preferred manner at least two pins arranged at a distance from each other are formed on the portion. This has the advantage that the co-operation of the pin and the set of teeth is likewise carried out in a protected manner. In addition, depending upon the method of production, the first or the further positioning device can be arranged on the cylindrical portion or the setting wheel.

In a further preferred embodiment of the present invention it would likewise be possible for the external set of teeth or the pin respectively to be provided on the peripheral face of the setting wheel and accordingly for the internal set of teeth or a pin respectively to be provided on at least one of the segments. In this case it is preferable for the respective set of teeth to co-operate with the pins.

In a further preferred embodiment of the present invention at least one tooth of the further positioning device has a flank which is made steeper as compared with the majority of the flanks. In this case “steeper” refers to the orientation of the flank with respect to the point at which the flank is in contact with the base or the setting wheel or the cylindrical portion respectively.

It is preferable for the flank to be arranged substantially at an angle of 90° to the base, as a result of which this flank restricts the rotational movement of the setting wheel. This is advantageous since an adjustment of the setting wheel is possible only in a region provided and, in this way, the twisting (removing/turn off) of the setting wheel for example is prevented.

In a further preferred embodiment of the present invention markings for determining the exit length of the lancet element out of the blood lancet apparatus are provided at least in part on the cap and the setting wheel. This is advantageous since a desired exit length of the lancet is capable of being set or controlled with reference to the markings in a simple manner.

Further advantages and embodiments may be seen in the accompanying drawings. In the drawings

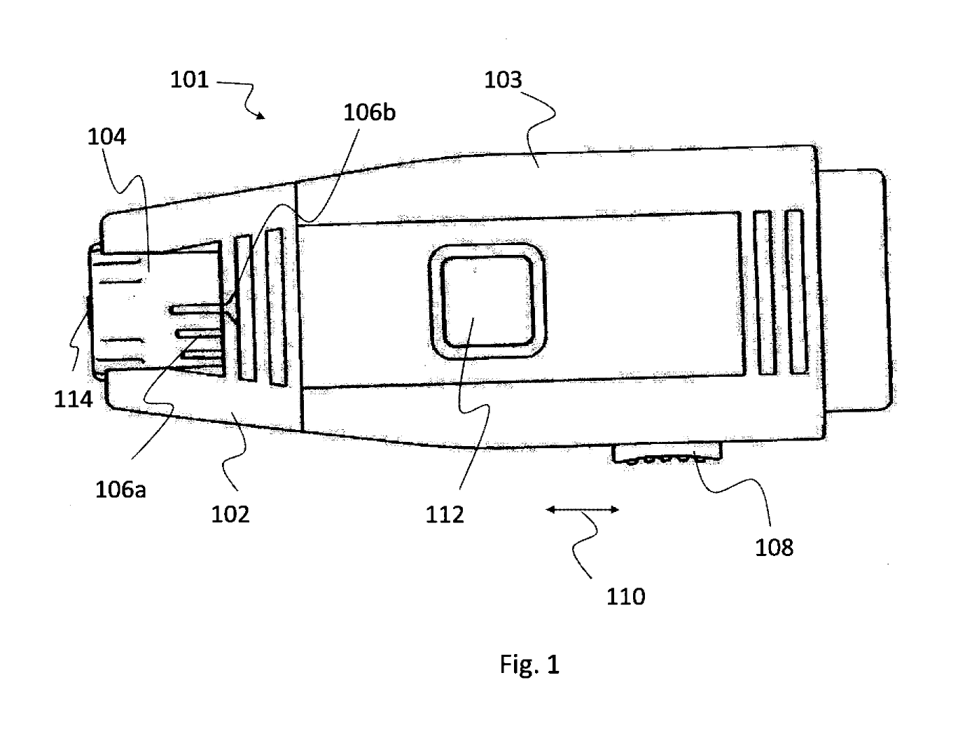

FIG. 1 is a front view of a blood lancet apparatus according to the invention;

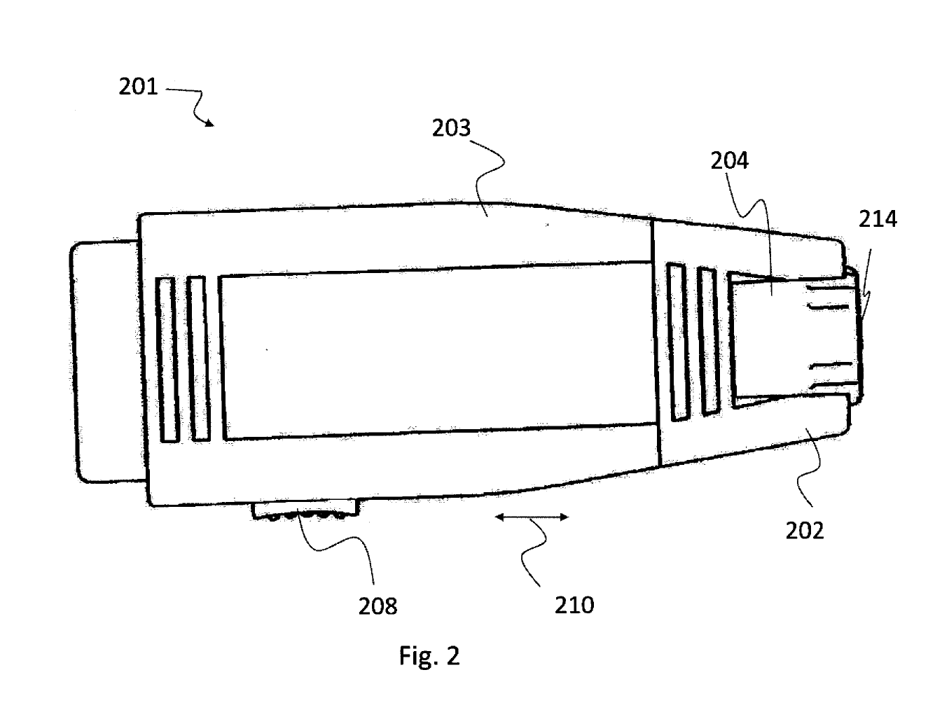

FIG. 2 is a rear view of a blood lancet apparatus according to the invention;

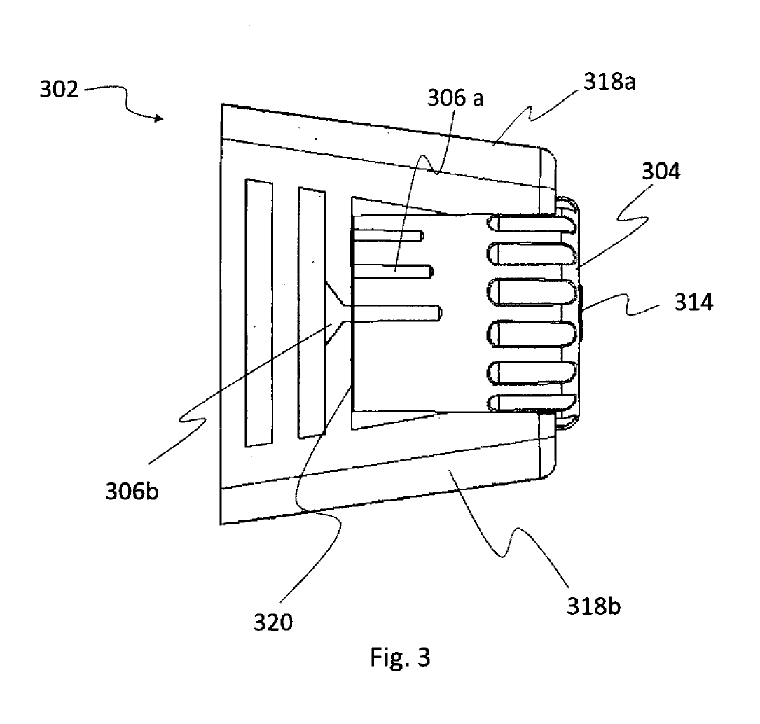

FIG. 3 is a plan view of the cap according to the invention and the setting wheel according to the invention in a first state;

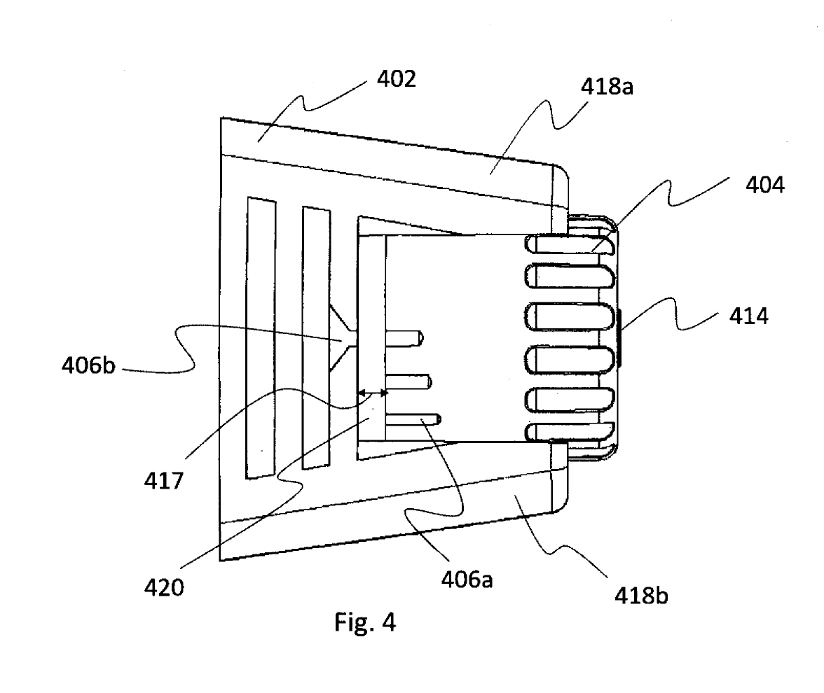

FIG. 4 is a plan view of the cap according to the invention and the setting wheel according to the invention in a second state;

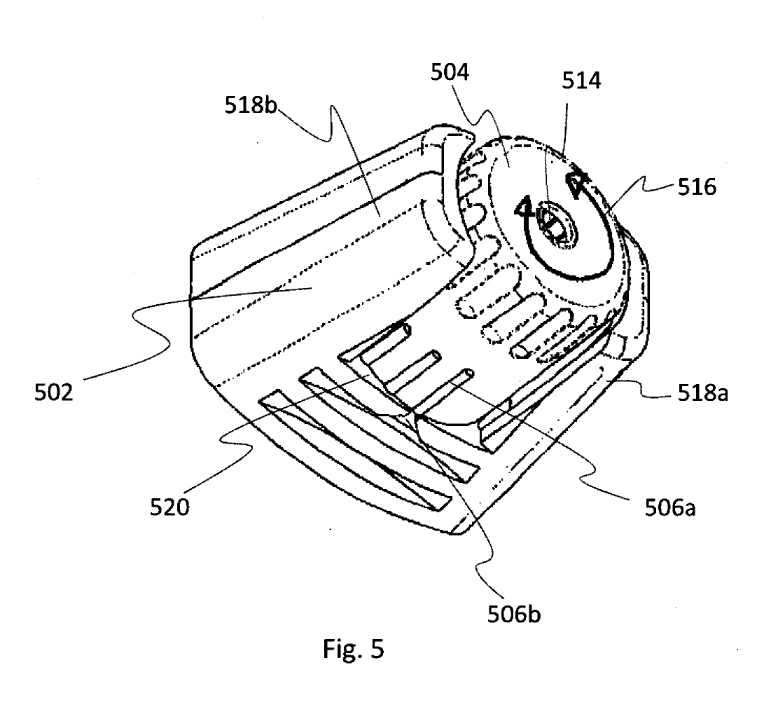

FIG. 5 is a perspective illustration of the cap according to the invention and the setting wheel according to the invention;

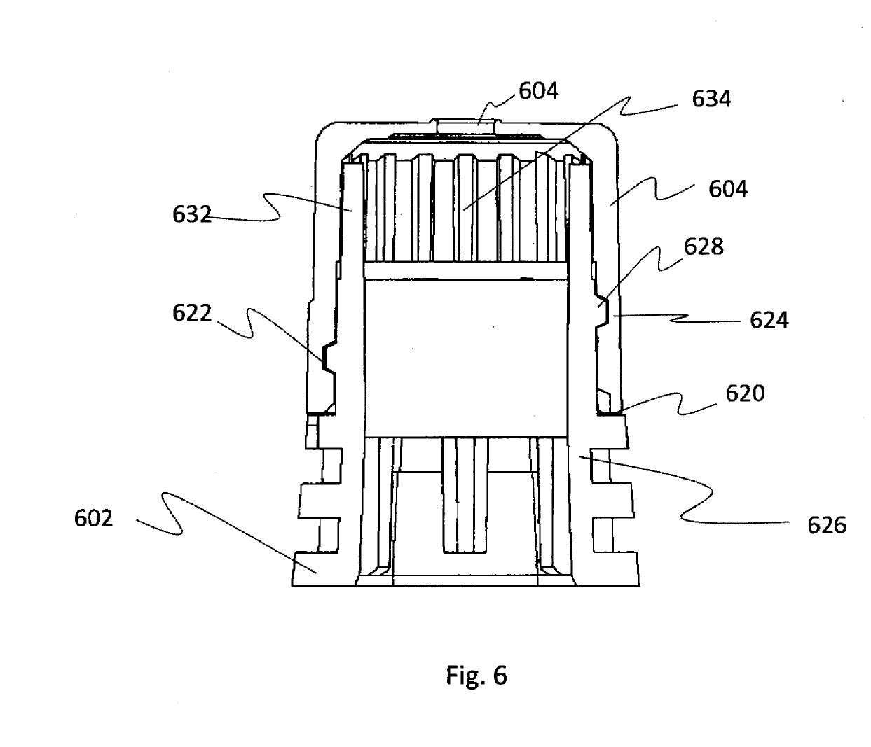

FIG. 6 is a first sectional illustration of a setting wheel according to the invention;

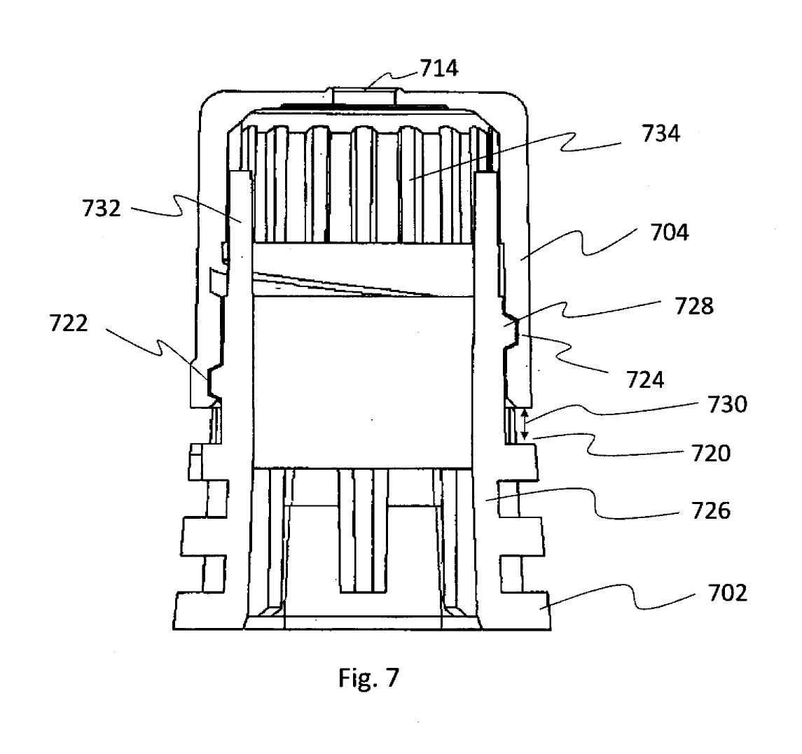

FIG. 7 is a second sectional illustration of a setting wheel according to the invention;

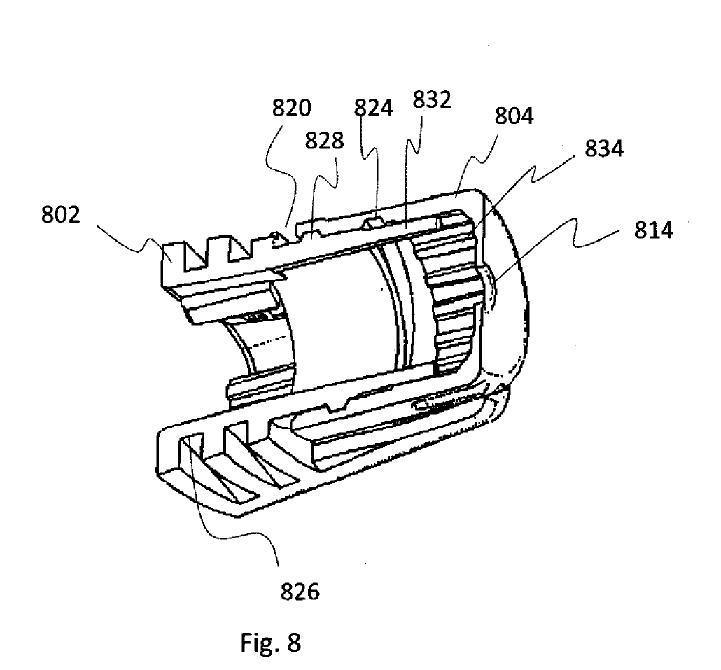

FIG. 8 is a perspective sectional illustration of a setting wheel according to the invention;

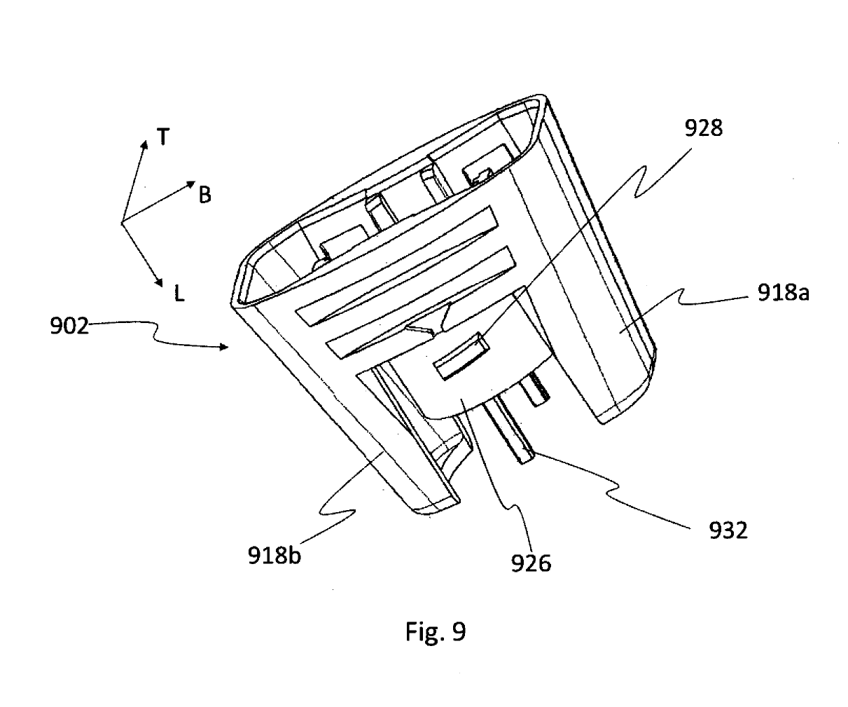

FIG. 9 is a perspective illustration of a cap according to the invention;

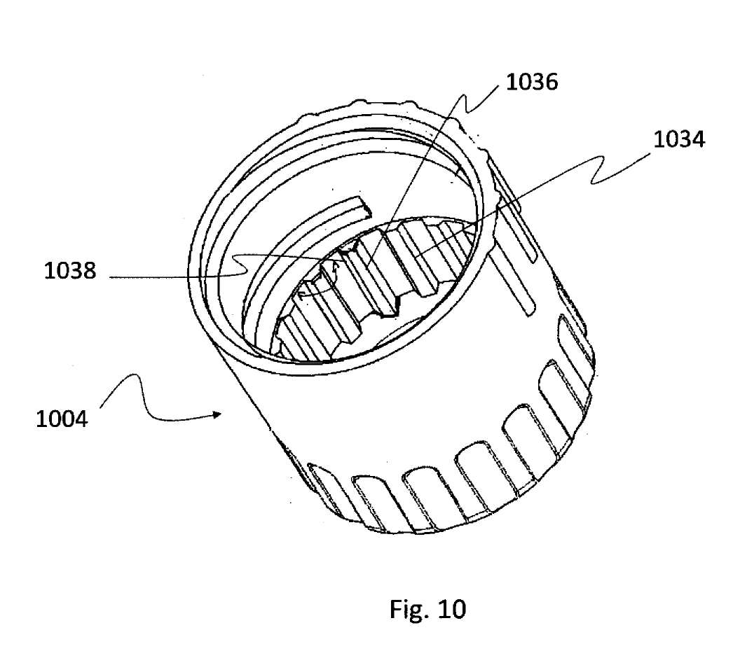

FIG. 10 is a perspective view of a setting wheel according to the invention;

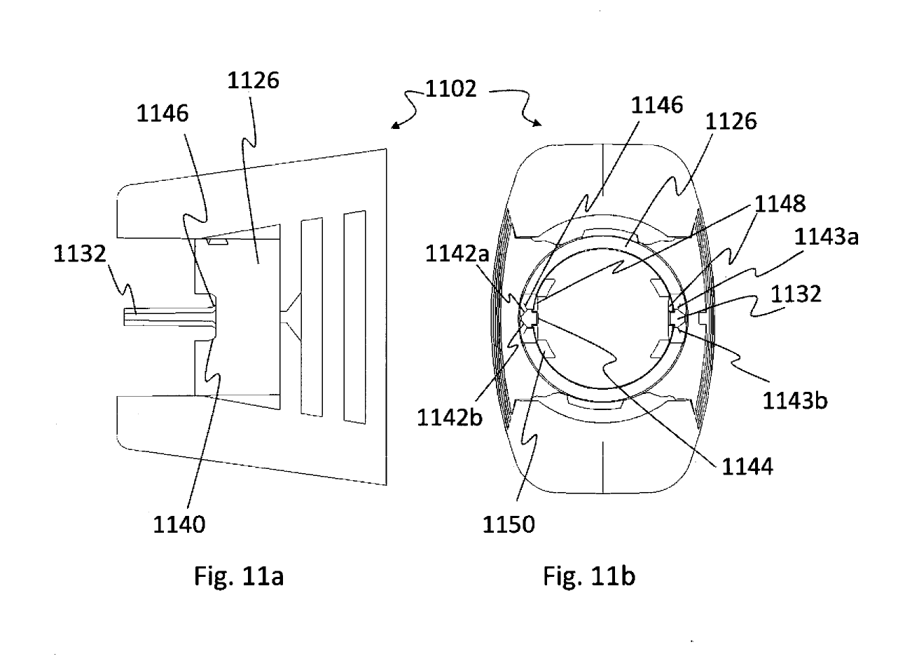

FIG. 11a is a plan view of a further cap according to the invention;

FIG. 11b is a front view of the further cap according to the invention;

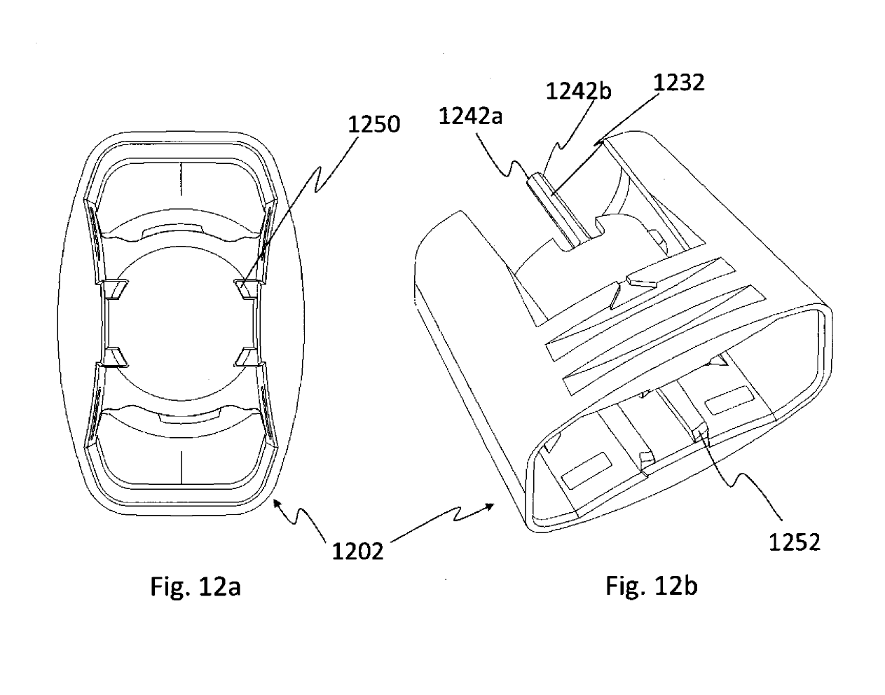

FIG. 12a is a rear view of the further cap according to the invention;

FIG. 12b is a perspective view of the cap according to the invention;

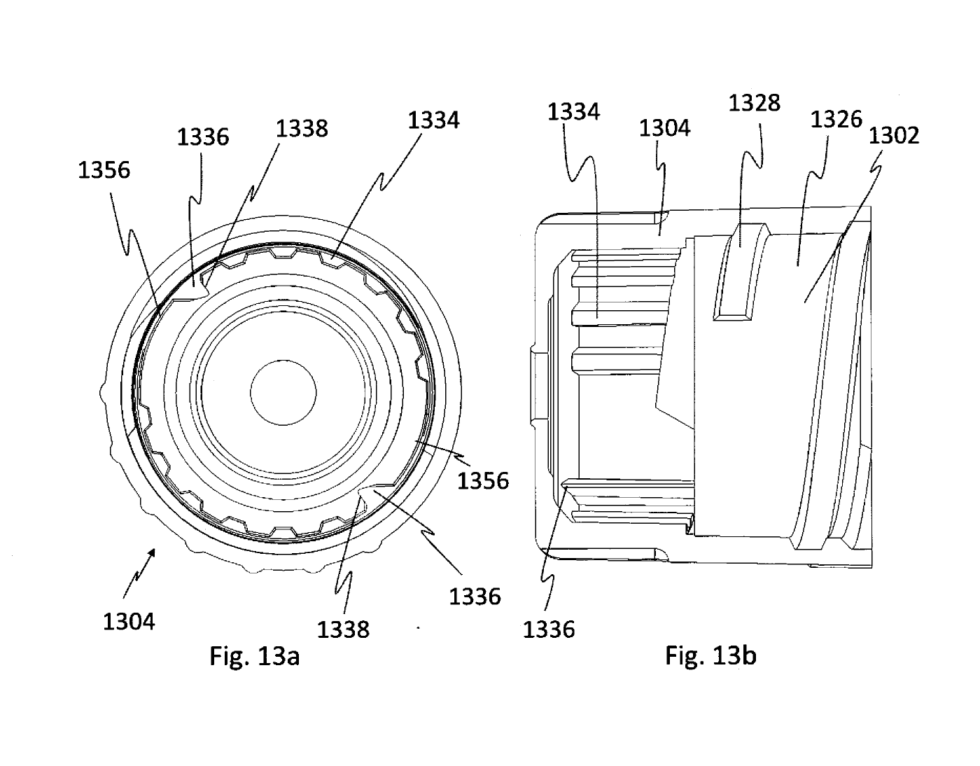

FIG. 13a is a sectional illustration of a further setting wheel according to the invention;

FIG. 13b is a sectional illustration of a setting wheel according to the invention coupled to the cap according to the invention;

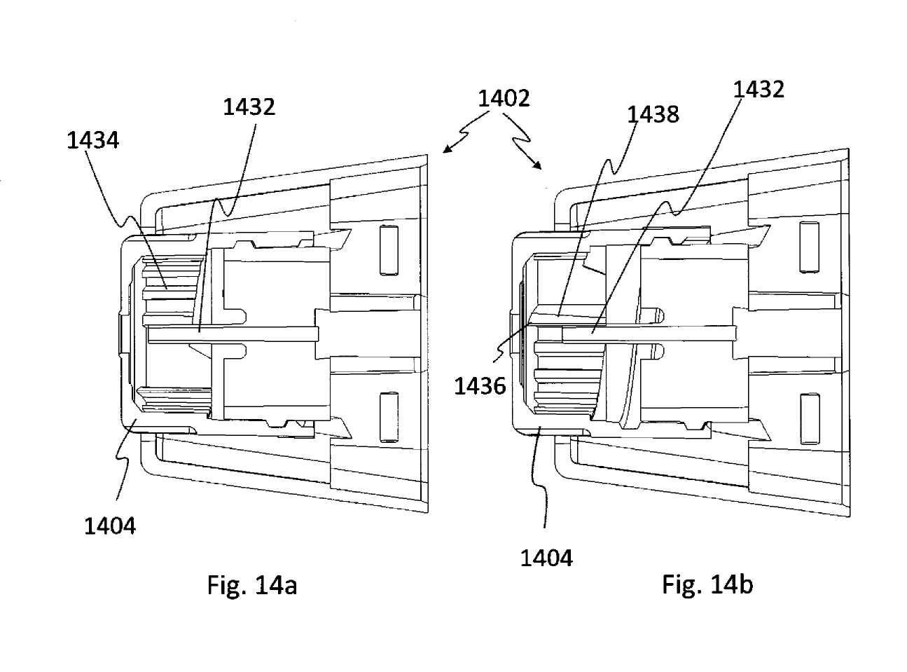

FIG. 14a is a sectional illustration of the cap according to the invention and the setting wheel according to the invention in a first state;

FIG. 14b is a sectional illustration of the cap according to the invention and the setting wheel according to the invention in a second state;

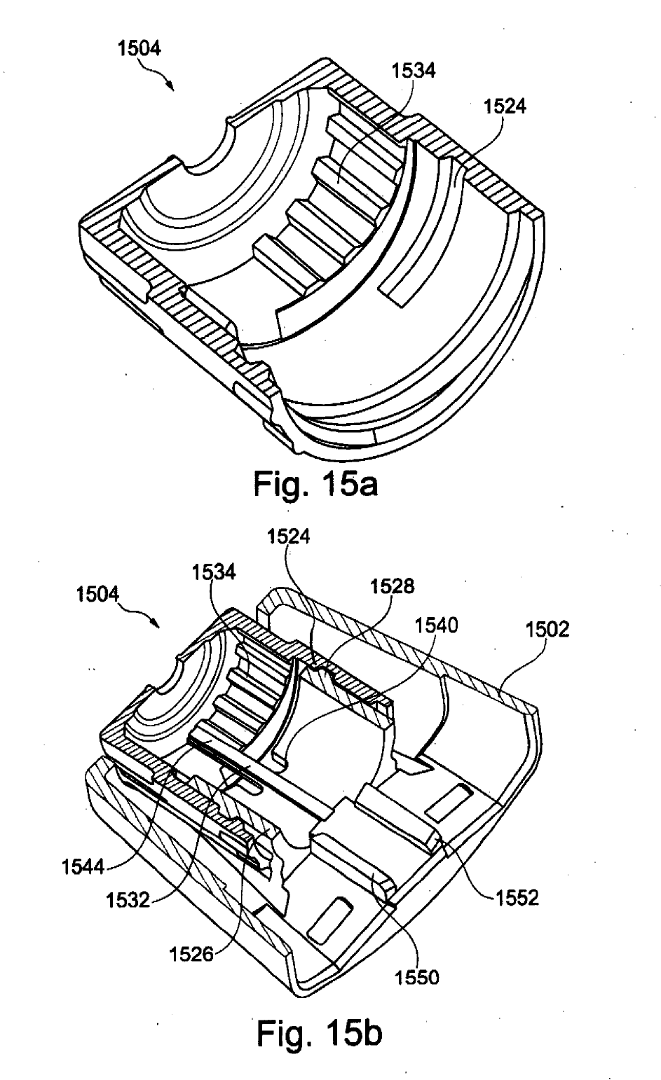

FIG. 15a is a three-dimensional sectional illustration of a setting wheel according to the invention;

FIG. 15b is a three-dimensional sectional illustration of a setting wheel according to the invention and a cap according to the invention in a coupled state, and

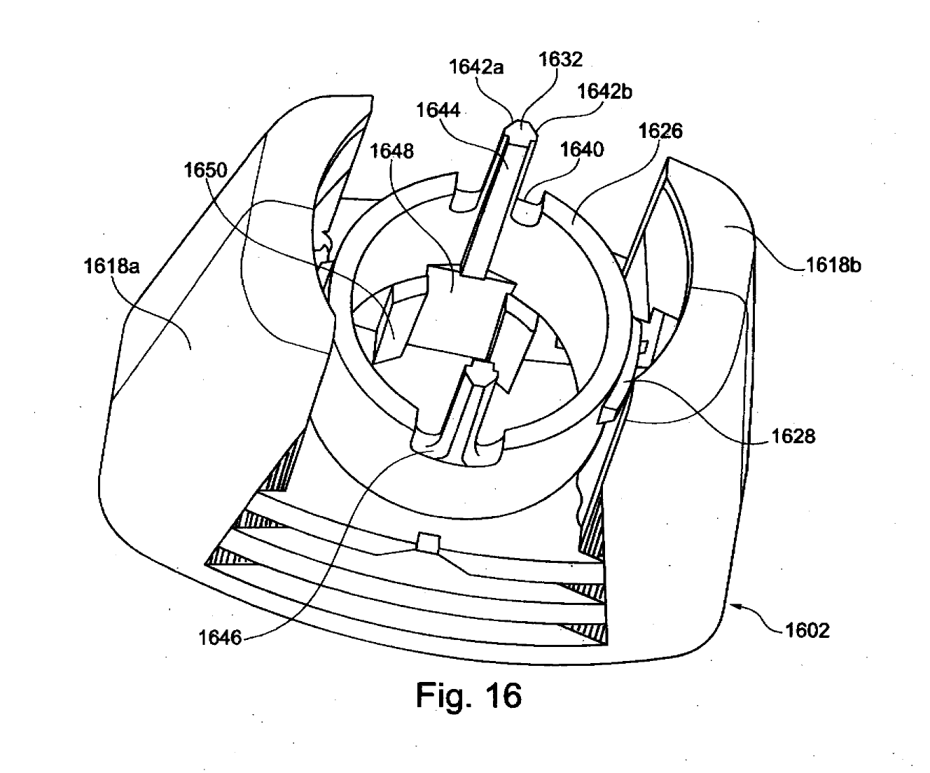

FIG. 16 is a three-dimensional illustration of the further cap according to the invention.

A blood lancet apparatus 101, 201 according to the invention is illustrated in FIG. 1 and FIG. 2. The blood lancet apparatus 101, 201 is shown from its front side in FIG. 1 and from its rear side in FIG. 2. The blood lancet apparatus 101, 201 has a cap 102, 202 which is coupled to a casing 103, 203. In addition, a setting wheel 104, 204, which is movable relative to the cap 102, 202 and which has markings 106a, 206a, is arranged on the cap 102, 202. Corresponding to the markings 106a, 206a of the setting wheel 104, 204 the cap 102, 202 likewise has at least one marking 106b, 206b. On the basis of the markings 106a, 206a and the at least one marking 106b, 206b an operator or a patient can carry out a setting of the pricking depth as desired or he or she can directly read off the configuration present in each case. As a result, the operator can make a choice of one desired pricking depth of preferably different pricking depths or exit lengths (L1, L2, L3) by a rotation of the setting wheel 104, 204. In this case the exit lengths (L1, L2, L3) can be formed in any desired staggered arrangement. Furthermore it is possible, depending upon requirements, for any desired of number exit lengths to be provided. A manual actuation element 108, 208 for actuating a slide element 109, 209, which is not shown in detail in these illustrations, is arranged at the side of the blood lancet apparatus 101, 201. This slide 109, 209 can be displaced in the direction of the double arrow 110, 210 in the longitudinal direction L of the blood lancet apparatus 101, 201. A press button 112, 212 arranged at the rear is used for pre-tensioning a lancet holder and a lancet inside the casing 103, 203 so as subsequently to carry out—by means of the press button 112, 212—the actual triggering procedure in which the lancet is moved out of an exit opening, 114, 214 for a short time and is preferably moved in again after that.

With reference to the setting of the pricking depth or the adjustment of the pricking depth respectively, a cap 302, 402, 502 with an associated setting wheel 304, 404, 504 is illustrated in FIGS. 3, 4 and 5, in which the cap 302, 402, 502 is arranged with the setting wheel 304, 404, 504 on the front side of the casing 303, 403, 503 of the blood lancet apparatus 301, 401, 501. This is evident from FIG. 1 and FIG. 2.

The cap 302, 402, 502 is characterized in that it does not embrace the setting wheel 304, 404, 504 completely, but extends forwards only laterally. As a result, it is advantageously possible for the setting wheel 304, 404, 504 to be turned in the direction of the double arrow 316, 416, 516, without an undesired displacement of the setting wheel 304, 404, 504 taking place, since a lateral embrace of the cap 302, 402, 502 by the segments 318a, 418a, 518a and 318b, 418b, 518b has taken place.

In FIG. 3 the setting wheel 304 is in a rotational position such that there is a maximum pricking/insertion depth of a lancet (not shown here) which projects out of the exit opening 314. In this position the setting wheel 304 is inserted or is turned to the maximum degree with respect to the cap 302, in such a way that in its rear region 320 it is at the shortest distance from the cap 302.

In FIG. 4, on the other hand, the setting wheel 404 is turned in such a way that it provides the smallest pricking depth of the lancet projecting out of the exit opening 414. In this position the setting wheel 404 is at a distance from the cap 402 in its rear region 420, as is evident from the reference number 417 which is intended to indicate the distance.

FIG. 5 is a perspective illustration of the arrangement shown in FIG. 3. In this case the cap 502 is movable in the direction of the double arrow 520.

In addition, the cap 602, 702, 802 for the setting of the pricking depth or the adjustment of the pricking depth respectively together with the setting wheel 604, 704, 804 is indicated in FIGS. 6 to 8. These are cross-sectional illustrations.

Furthermore, it is evident from FIGS. 6 to 8 that the setting wheel 604, 704, 804 can be turned with respect to the cap 602, 702, 802 by means of a thread arrangement 622, 722, 822 illustrated diagrammatically, the setting wheel 604, 704, 804 having an internal threaded portion 624, 724, 824 and the cylindrical portion 626, 726, 826 of the cap 602, 702, 802 having an external threaded portion 628, 728, 828, so that in FIG. 6 there is a rotational position which ensure the highest pricking depth of the lancet.

In FIG. 7 a rotational position of the setting wheel 704 is shown which indicates a smallest pricking depth. In the state of the smallest pricking depth a distance 730 is present between the setting wheel 704 and the cap 702.

A perspective illustration of the arrangement shown in FIG. 7 is indicated in FIG. 8.

In FIG. 9 and FIG. 10 the cap 902 or the setting wheel 1004 respectively is illustrated with pins 932, 1032 integrated on it, which have the task of engaging in a structure 934, 1034—situated on the inside in the manner of a projection—on the cap 902 or on the setting wheel 1004. This is intended to permit a sort of “ratchet effect” in order to ensure a step-wise adjustment of the setting wheel 1004 with respect to the cap 902. The arrow B represents the width-wise direction or the first cross-sectional direction respectively of the cap and the arrow T represents the depth direction or the second cross-sectional direction respectively of the cap.

In the same way, a tooth 936, 1036 of this ratchet-like structure 934, 1034 is designed in such a way that it has a higher flank 938, 1038 on one or both sides. As a result, a sort of stop is obtained, so that the setting wheel 1004 can be turned only as far as a specified maximum rotation. This ensures that the setting wheel 1004 cannot be turned off in an unintentional manner.

A further embodiment of the cap 1102 is illustrated in FIGS. 11a and 11b, in which the pins 1132 are arranged in a recess 1140 in the cylindrical portion 1126. In this case the foot 1146 of the pin or the transition from the pin 1132 to the cylindrical portion 1126 is preferably made curved, this having the advantage that as a result of a deflection of one of the pins 1132 no notching effect or a considerably reduced notch effect occurs in the connecting point between the pin 1132 and the cylindrical portion 1126; this leads to a greater strength of the pin 1132 or to an increase in the service life respectively.

As is evident from FIG. 11b, the pins 1132 have flanks 1142a and 1142b which are inclined with respect to the back 1144 of the pin. The teeth of the ratchet-like structure of the setting wheel (not shown) can slide on the flanks 1142a and 1142b. The lateral regions 1143a and 1143b of the pins 1132 can be used with a tooth (not shown) for blocking the rotational movement of a setting wheel. In this case it is also possible, however, for the tooth for blocking the rotational movement to co-operate or come into contact with one of the lateral regions 1143a and 1143b and/or with one of the flanks 1142a and 1142b.

It is likewise possible for two teeth to co-operate at the same time with two pins 1132. The back 1148 of the pin can likewise extend at least in part over the cylindrical portion 1126 in the longitudinal direction L and it can be connected to or co-operate with a base 1148 of the pin, as a result of which the stability 1132 of the pin 1132 and the entire cap 1102 is increased.

The rod-shaped elements 1150 can likewise contribute to the stabilization of the cap 1102. Furthermore, it is possible for these elements 1150 to be used for the coupling of the cap 1102 to a casing or base member or to form a guide for a lancet element or a lancet element receiving means.

A cap 1202 is likewise shown in FIGS. 12a and 12b. The flanks 1242a and 1242b of the pins 1232 can be seen from the perspective illustration of FIG. 12b. Furthermore, it is evident from the illustrations that the rod-shaped elements 1250 can be designed with inclined faces 1252, a structure of this type facilitating the co-operation of the cap 1202 with a casing for example.

FIGS. 13a and 13b show a setting wheel 1304 which has a ratchet-like structure 1334 which preferably comprises a plurality of teeth formed in the same way. In this case the ratchet-like structure 1334 can extend over the entire internal periphery of the setting wheel 1304, it being preferable for only one or, in a particularly preferred manner, two portions of the internal periphery to be provided with the ratchet-like structure 1334 and for the sum of the portions to correspond to a portion of the periphery which is preferably smaller than the internal periphery of the setting wheel 1304. Regions 1356 which are preferably uniform and which extend in the peripheral direction are thus provided between the ratchet-like structures 1334. This embodiment has the advantage that the setting wheel 1304 is capable of being produced in a simpler and more rapid manner as compared for example with an embodiment in which the ratchet-like design 1334 extends over the entire periphery.

A tooth 1336 of one or preferably two ratchet-like structures 1334 has a higher and/or steeper flank 1338. Sliding of the pin is prevented by the co-operation of the flank 1338 with a pin of the cap 1302, as a result of which the resilient pin is not capable of being deflected, i.e. a rotational movement of the setting wheel 1304 is blocked.

Furthermore, an external threaded portion 1328 of the cap 1302, which in this illustration extends in a one-piece form around the cylindrical portion 1326, is shown in FIG. 13b.

Two states of a coupled setting wheel/cap arrangement are illustrated in FIGS. 14a and 14b. A state in which a lancet element can emerge further out of the setting wheel 1404 as compared with the state shown in FIG. 14b is shown in FIG. 14a.

It is evident from the two illustrations (14a and 14b) that the ratchet-like structure 1434 has teeth which are of different length. The individual teeth of the ratchet-like structure 1434 are designed in their length in such a way that during a turning of the setting wheel 1404 and the displacement of the setting wheel 1404 resulting therefrom in the longitudinal direction L constantly comparable surface portions of the ratchet-like structure 1434 are in contact with the pin 1432. It is preferable for the surface portions always to be equally large since on account of an arrangement of this type the force required to turn the setting wheel 1404 is always the same or the same force pattern is always present. This means that an altered force pattern is preferably present only when the pins 1432 co-operate with a flank 1438 of the tooth 1436.

Three-dimensional sectional illustrations of the setting wheel 1504 and the cap 1502 are reproduced in FIGS. 15a and 15b. It is evident that the ratchet-like structure 1534 has teeth which are of different length or the length of the teeth is different in a manner dependent upon the position provided in the peripheral direction in each case. It is preferable for a linear ratio to exist between the length of the teeth and the respective position in the peripheral direction. It is particularly preferred for the length of the individual teeth to be dependent upon the pitch of the internal thread 1524 or the external thread 1528. Furthermore, it is possible for a curve which extends parallel to the thread to be formed by the teeth of different length.

In addition, it is evident from FIG. 15b that the back 1544 of the pin extends on the pin 1532 and the cylindrical portion 1526. In this case the pin 1532 is arranged in a recess 1540 in the cylindrical portion 1526. In this illustration (FIG. 15b) the rod-shaped elements 1550 have inclined faces 1552 by which a co-operation of the cap 1502 with further devices is facilitated or made possible respectively.

A cap 1602 is shown in a three-dimensional illustration in FIG. 16. This cap 1602 has two segments 1618a and 1618b, between which a cylindrical portion 1626 is arranged. A threaded portion 1628, which can co-operate with an internal thread of a setting wheel, is provided on the cylindrical portion 1626.

Two pins 1632 are provided in each case in different recess 1640. In this case each pin 1632 has an arrow-shaped cross-section. The tip of the arrow-shaped cross-section is formed at least by two flanks 1642a and 1642b, in which case the ends of the flanks orientated in the direction of the tip can be in contact with each other, but they preferably terminate at a portion opposite the back 1644 of the pin. In this case this portion can be made parallel to the back 1644 of the pin. As shown in this figure, the pins 1632 can in each case have a foot 1646 of pin which forms a curve-shaped transition from the pin 1632 to the cylindrical portion 1626.

The back 1644 of the pin extends over the entire pin 1632 and at least locally over the cylindrical portion 1626 as far as a base 1648 of the pin which projects at least locally into the space formed by the cylindrical portion 1626.

All the features disclosed in the application documents are claimed as being essential to the invention, insofar as they are novel either individually or in combination as compared with the prior art.

### LIST OF REFERENCES

| | |
|---|---|
| 101, 201 | blood lancet apparatus |
| 102, 202, 302, 402, 502, 602, | cap |
| 702, 802, 902, | |
| 1102, 1202, 1302, 1402, 1502, | casing or base |
| 1602, 103, 203, | member respectively |
| 104, 204, 304, 404, 504, 604, | setting wheel |
| 704, 804, 1004, | |
| 1304, 1404, 1504 | |
| 106a, 306a, 406a, 506a | markings |
| 106b, 306b, 406b, 506b | markings |
| 108, 208 | hand actuation element |
| 110, 210 | double arrow (FIG. 1 |
| | and FIG. 2) |
| 112 | push button |
| 114, 214, 314, 414, 514, 614, | exit opening |
| 714, 814 | |
| 516 | double arrow (FIGS. |
| | 3 to 5) |
| 417 | distance |
| 318a, 418a, 518a, 918a, 1618a | segment |
| 318b, 418b, 518b, 918b, 1618b | segment |
| 320, 420, 520, 620, 720, 820 | rearward region |
| 622, 722, 822 | thread arrangement |
| 624, 724, 824, 1524 | internally threaded |
| | portion |
| 626, 726, 826, 926, 1126, 1326, | cylindrical portion |
| 1526, 1626 | |
| 628, 728, 828, 928, 1328, 1528, | externally threaded |
| 1628 | portion |
| 730 | distance |
| 632, 732, 832, 932, 1132, 1232, | pin |
| 1432, 1532, 1632 | |
| 1034, 1334, 1434, 1534 | ratchet-like structure |
| 1036, 1336, 1436 | tooth |
| 1038, 1338, 1438 | higher flank |
| 1140, 1540, 1640 | recess |
| 1142a/b, 1242a/b, 1642a/b | flank of the pin |
| 1143 a/b | lateral regions of the |
| | pin |
| 1144, 1544, 1644 | back of the pin |
| 1146, 1646 | foot of the pin |
| 1148, 1648 | base of the pin |
| 1150, 1250, 1550, 1650 | rod-shaped elements |
| 1252, 1552 | inclined face |
| 1356 | uniform region |
| L | longitudinal direction |
| B | first cross-sectional |
| | direction or width- |
| | wise direction |
| T | second cross-sectional |
| | direction or depth |
| | direction |

1. A blood lancet apparatus with a base member (103, 203, 303, 403, 503) for the arrangement of apparatus components, a lancet-receiving device for holding at least one lancet element, which is surrounded at least locally by the base member (103, 203, 303, 403, 503), and a cap (102, 202, 302, 402, 502, 602, 702, 802, 902, 1102, 1202, 1302, 1402, 1502, 1602) coupled to the base member for the arrangement of a setting wheel (104, 204, 304, 404, 504, 604, 704, 804, 1004, 1304, 1404, 1504) on a portion (626, 726, 826) of the cap which is cylindrical at least in part, in order to set a plurality of exit lengths (L1, L2, L3) of the lancet element out of the blood lancet apparatus (101, 201, 301, 401, 501), wherein the setting wheel (104, 204, 304, 404, 504, 604, 704, 804, 1004, 1304, 1404, 1504) has an internally threaded portion (624, 724, 824, 1524) and the cylindrical portion (626, 726, 826) is provided with an externally threaded portion (628, 728, 828, 1328, 1528, 1628), wherein the threaded portions (624, 724, 824, 1524, 628, 728, 828, 1328, 1528, 1628) are connected to one another functionally in such a way that the setting wheel is rotatable and adjustable in the longitudinal direction (L) of the base member, characterized in that the setting wheel (104, 204, 304, 404, 504, 604, 704, 804, 1004, 1304, 1404, 1504) is surrounded by arm-like segments (318a, 418a, 518a, 1618a, 318b, 418b, 518b, 1618b) extending in the longitudinal direction (L), in such a way that an actuation of the setting wheel required for setting the exit length is capable of being carried out only in the segment-free peripheral portions.

2. A blood lancet apparatus according to claim 1, characterized in that the cap (102, 202, 302, 402, 502, 602, 702, 802, 902, 1102, 1202, 1302, 1402, 1502, 1602) and/or the base member (103, 203, 303, 403, 503) has or have a cross-section which in a first cross-sectional direction (B) is greater than the diameter of the setting wheel (104, 204, 304, 404, 504, 604, 704, 804, 1004, 1304, 1404, 1504) and in a second cross-sectional direction (T) is smaller than the cross-section in the cross-sectional first direction (B).

3. A blood lancet apparatus according to claim 1 or 2, characterized in that the cylindrical portion of the cap (102, 202, 302, 402, 502, 602, 702, 802, 902, 1102, 1202, 1302, 1402, 1502, 1602) is arranged between the two arm-like segments (318a, 418a, 518a, 1618a, 318b, 418b, 518b, 1618b) of the cap (102, 202, 302, 402, 502, 602, 702, 802, 902, 1102, 1202, 1302, 1402, 1502, 1602) enclosing the portion (102, 202, 302, 402, 502, 602, 702, 802, 902, 1102, 1202, 1302, 1402, 1502, 1602) in part.

4. A blood lancet apparatus according to claim 3, characterized in that the segments (318a, 418a, 518a, 1618a, 318b, 418b, 518b, 1618b) are made uniform.

5. A blood lancet apparatus according to claim 3 or 4, characterized in that the segments are arranged in an opposed manner with respect to the cylindrical portion.

6. A blood lancet apparatus according to any one of claims 3 to 5, characterized in that the setting wheel (104, 204, 304, 404, 504, 604, 704, 804, 1004, 1304, 1404, 1504) is longer than the segments (318a, 418a, 518a, 1618a, 318b, 418b, 518b, 1618b) or projects at least in part in the longitudinal direction (L) with respect to the segments.

7. A blood lancet apparatus according to claim 6, characterized in that a first positioning device (932, 1032, 1132, 1232, 1432, 1532, 1632) and a further positioning device (934, 1034, 1334, 1434, 1534) extend at least in part in the longitudinal direction (L), and the first positioning device (932, 1032, 1132, 1232, 1432, 1532, 1632) comprises at least one pin (932, 1032, 1132, 1232, 1432, 1532, 1632) and the further positioning device (934, 1034, 1334, 1434, 1534) is made corrugated, in particular serrated/tooth like, wherein the at least one pin (932, 1032, 1132, 1232, 1432, 1532, 1632) is capable of being deflected at least for a time.

8. A blood lancet apparatus according to claim 7, characterized in that the further positioning device (934, 1034, 1334, 1434, 1534) surrounds at least in part the cylindrical portion (626, 726, 826) and has an external set of teeth (934, 1034, 1334, 1434, 1534) and the teeth have tooth flanks, of which the number is substantially equal.

9. A blood lancet apparatus according to claim 8, characterized in that at least one pin (932, 1032, 1132, 1232, 1432, 1532, 1632) is formed on the setting wheel (104, 204, 304, 404, 504, 604, 704, 804, 1004, 1304, 1404, 1504).

10. A blood lancet apparatus according to claim 8, characterized in that the first positioning device (932, 1032, 1132, 1232, 1432, 1532, 1632) is surrounded at least in part by the setting wheel (104, 204, 304, 404, 504, 604, 704, 804, 1004, 1304, 1404, 1504), the setting wheel has at least in part an internal set of teeth (934, 1034, 1334, 1434, 1534) and the teeth have tooth flanks, of which the number is substantially equal.

11. A blood lancet apparatus according to claim 10, characterized in that a pin (932, 1032, 1132, 1232, 1432, 1532, 1632) is formed on the—at least in part—cylindrical portion (626, 726, 826), and at least two pins (932, 1032, 1132, 1232, 1432, 1532, 1632) arranged at a distance from each other are preferably formed on the portion.

12. A blood lancet apparatus according to any one of claims 7 to 11, characterized in that at least one tooth (936, 1036, 1336, 1436) of the further positioning device (934, 1034, 1334, 1434, 1534) has a flank which is made steeper as compared with the majority of the flanks, wherein the rotational movement of the setting wheel (104, 204, 304, 404, 504, 604, 704, 804, 1004, 1304, 1404, 1504) is restricted by this flank.

13. A blood lancet apparatus according to any one of the preceding claims, characterized in that the cap (102, 202, 302, 402, 502, 602, 702, 802, 902, 1102, 1202, 1302, 1402, 1502, 1602) and the setting wheel (104, 204, 304, 404, 504, 604, 704, 804, 1004, 1304, 1404, 1504) have at least in part markings (106a, 206a, 106b, 206b) for determining the exit length of the lancet element.
